(19) United States
(12) Patent Application Publication
(10) Pub. No.: US 2005/0070544 A1
(43) Pub. Date: Mar. 31, 2005

(54) 1,2,5,10-tetrahydropyridazino{4,5-b}quinoline-1,10-diones and their use for the treatment of pain

(75) Inventors: Megan Murphy, Wilmington, DE (US); Wenhua Xiao, Wilmington, DE (US); Dean Gordon Brown, Wilmington, DE (US); Rebecca Ann Urbanek, Wilmington, DE (US); Frances Marie MClaren, Wilmington, DE (US); Edward Vacek, Wilmington (DE); Thomas Lynn Bare, West Chester (DE); Carey Lynn Horchler, Wilmington, DE (US); Christine Barlaam, Reims (FR); Gary Banks Steelman, Wilmington, DE (US); Vernon Alford, Raritan, NJ (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB

(21) Appl. No.: 10/381,914

(22) PCT Filed: Sep. 28, 2001
(86) PCT No.: PCT/SE01/02124

Related U.S. Application Data
(60) Provisional application No. 60/236,754, filed on Sep. 29, 2000.

(51) Int. Cl. A61K 31/503; C07D 487/02

(52) U.S. Cl. 514/248; 544/234

(57) ABSTRACT

Compounds are disclosed according to structural diagram (1), wherein R¹, A, D and E are as defined in the specification. Also disclosed are methods for the treatment of pain and pharmaceutical compositions comprising a pain-ameliorating effective amount of a compound in accord with structural diagram (1).

### FIELD OF THE INVENTION

This invention relates to the treatment or prevention of pain or nociception.

### RELATED ART

Pain is a sensory experience distinct from sensations of touch, pressure, heat and cold. It is often described by sufferers by such terms as bright, dull, aching, pricking, cutting or burning and is generally considered to include both the original sensation and the reaction to that sensation. This range of sensations, as well as the variation in perception of pain by different individuals, renders a precise definition of pain difficult, however, many individuals suffer with severe and continuous pain.

Pain that is caused by damage to neural structures is often manifest as a neural supersensitivity or hyperalgesia and is termed “neuropathic” pain. Pain can also be “caused” by the stimulation of nociceptive receptors and transmitted over intact neural pathways, such pain is termed “nociceptive” pain.

The level of stimulation at which pain becomes noted is referred to as the “pain threshold.” Analgesics are pharmaceutical agents which relieve pain by raising the pain threshold without a loss of consciousness. After administration of an analgesic drug a stimulus of greater intensity or longer duration is required before pain is experienced. In an individual suffering from hyperalgesia an analgesic drug may have an anti-hyperalgesic effect. In contrast to analgesics, agents such as local anaesthetics block transmission in peripheral nerve fibers thereby blocking awareness of pain. General anaesthetics, on the other hand, reduce the awareness of pain by producing a loss of consciousness.

Tachykinin antagonists have been reported to induce antinociception in animals, which is believed to be analogous to analgesia in man (Maggi et al, J. Auton. Pharmacol. (1993) 13, 23-93). In particular, non-peptide NK-1 receptor antagonists have been shown to produce such analgesia. For example, the NK-1 receptor antagonist RP 67,580 produced analgesia with potency comparable to that of morphine (GarTet et al, Proc. Natl. Acad. Sci. USA (1993) 88, 10208-10212).

The opioid analgesics are a well-established class of analgesic agents with morphine-like actions. Synthetic and semi-synthetic opioid analgesics are derivatives of five chemical classes of compound: phenanthrenes; phenylheptylamines; phenylpiperidines; morphinans; and benzomorphans. Pharmacologically these compounds have diverse activities, thus some are strong agonists at the opioid receptors (e.g. morphine); others are moderate to mild agonists (e.g. codeine); still others exhibit mixed agonist-antagonist activity (e.g. nalbuphine); and yet others are partial agonists (e.g. nalorphine). Whilst an opioid partial agonist such as nalorphine, (the N-alkyl analogue of morphine) will antagonize the analgesic effects of morphine, when given alone it can be a potent analgesic in its own right.

Of all of the opioid analgesics, morphine remains the most widely used, but, in addition to its therapeutic properties, it has a number of drawbacks including respiratory depression, decreased gastrointestinal motility (resulting in constipation), nausea and vomiting. Tolerance and physical dependence also limit the clinical uses of opioid compounds.

Aspirin and other salicylate compounds are frequently used in treatment to interrupt amplification of the inflammatory process in rheumatoid diseases and arthritis and temporarily relieve the pain. Other drug compounds used for these purposes include phenylpropionic acid derivatives such as Ibuprofen and Naproxen, Sulindac, phenyl butazone, corticosteroids, antimalarials such as chloroquine and hydroxychloroquine sulfate, and fenemates (J. Hosp. Pharm., 36:622 (May 1979)). These compounds, however, are ineffective for neuropathic pain.

Available therapies for pain also have drawbacks. Some therapeutic agents require prolonged use before an effect is experienced by the patient. Other existing drugs have serious side effects in certain patients, and subjects must be carefully monitored to ensure that any side effects are not unduly threatening. Most existing drugs provide only temporary relief from pain and must be taken consistently on a daily or weekly basis. With disease progression the amount of medication needed to alleviate the pain often increases, thus increasing the potential for adverse side effects.

NOVA receptors are defined by the binding of N-methyl-D-aspartate (NMDA) comprise a receptor/ion channel complex with several different identified binding domains. NMDA itself is a molecule structurally similar to glutamate (Glu) which binds at the glutamate binding suite and is highly selective and potent in activating the NMDA receptor (Watkins (1987); Olney (1989)).

Many compounds are known that bind at the NMDA/Glu binding site (for example CPP, DCPP-ene, CGP 40116, CGP 37849, CGS 19755, NPC 12626, NPC 17742, D-AP5, D-AP7, CGP 39551, CGP43487, MDL-100,452, LY-274614, LY-233536, and LY233053). Other compounds, referred to as non-competitive NMDA antagonists, bind at other sites in the NMDA receptor complex (examples are phencyclidine, dizocilpine, ketamine, tiletamine, CNS 1102, dextromethorphan, memantine, kynurenic acid, CNQX, DNOX, 6,7-DCQX, 6,7-DCHQC, R(+)-HA-966, 7-chloro-kynurenic acid, 5,7-DCKA, 5-iodo-7-chloro-kynurenic acid, MDL-28,469, MDL-100,748, MDL-29,951, L-689,560, L-687,414, ACPC, ACPCM, ACPCE, arcaine, diethylenetriiamine, 1,10-diaminodecane, 1,12-diaminododecane, ifenprodil, and SL-82.0715). These compounds have been extensively reviewed by Rogawski (1992) and Massieu et. al., (1993), and articles cited therein.

In addition to its physiological function, glutamate (Glu) can be neurotoxic. Glu neurotoxicity is referred to as “excitotoxicity” because the neurotoxic action of Glu, like its beneficial actions, is mediated by an excitatory process (Olney (1990); Choi (1992)). Normally, when Glu is released at a synaptic receptor, it binds only transiently and is then rapidly removed from the receptor by a process that transports it back into the cell. Under certain abnormal conditions, including stroke, epilepsy and CNS trauma, Glu uptake fails and Glu accumulates at the receptor resulting in a persistent excitation of electrochemical activity that leads to the death of neurons that have Glu receptors. Many neurons in the CNS have Glu receptors, so excitotoxicity can cause an enormous amount of CNS damage.

Acute excitotoxicity injury can occur as a result of ischemic events, hypoxic events, trauma to the brain or spinal cord, certain types of food poisoning which involve an excitotoxic poison such as domoic acid, and seizure-mediated neuronal degeneration, which can result from persistent epileptic seizure activity (status epilepticus). A large body of evidence has implicated the NMDA receptor as one receptor subtype through which Glu mediates a substantial amount of CNS injury, and it is well established that NMDA antagonists are effective in protecting CNS neurons against excitotoxic degeneration in these acute CNS injury syndromes (Choi (1988); Olney (1990)).

In addition to neuronal damage caused by acute insults, excessive activation of Glu receptors may also contribute to more gradual neurodegenerative processes leading to cell death in various chronic neurodegenerative diseases, including Alzheimer's disease, amyotrophic lateral sclerosis, AIDS dementia, Parkinson's disease and Huntington's disease (Olney (1990)). It is generally considered that NMDA antagonists may prove useful in the therapeutic management of such chronic diseases.

In the 1980's it was discovered that PCP (also known as “angel dust”) acts at a “PCP recognition site” within the ion channel of the NMDA Glu receptor. PCP acts as a non-competitive antagonist that blocks the flow of ions through the NMDA ion channel. More recently it has become evident that drugs which act at the PCP site as non-competitive NMDA antagonists are likely to have psychotomimetic side effects. Further, it is now recognized that certain competitive and non-competitive NMDA antagonists can cause similar pathomorphological effects in rat brain (Olney et. al., (1991); Hargreaves et. al., (1993)). Such compounds also have psychotomimetic effects in humans (Kristensen et. al., (1992); Herrling (1994); Grotta (1994)).

The glycine binding site of the NMDA receptor complex is distinguishable from the Glu and PCP binding sites. Also, it has recently been discovered that NMDA receptors occur as several subtypes which are characterized by differential properties of the glycine binding site of the receptor. Many compounds that bind at the NMDA receptor glycine site, useful for the treatment of stroke and neurodegenerative conditions, have been described in U.S. Pat. Nos. 5,604,227; 5,733,910; 5,599,814; 5,593,133; 5,744,471; 5,837,705 and 6,103,721.

### SUMMARY OF THE INVENTION

It has now been discovered that certain compounds which exhibit the property of binding to the NMDA receptor glycine site have utility for the amelioration of pain and particularly for the amelioration of neuropathic pain.

In a first aspect the invention provides compounds useful for the treatment of pain according to structural diagram I, 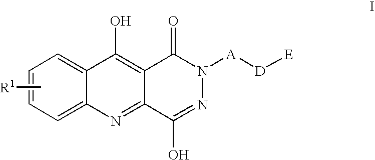
wherein: R¹ is halo; A is (CH₂)ₙC≡C where n is a value selected from 1, 2 or 3; D is aryl or heteroaryl; E is hydrogen or halogen, with the proviso that said compound of structural diagram I is not 7-chloro-4-hydroxy-2-[3-(phenyl)prop-2-ynyl]-1,2,5; 10-tetrahydropyridazino[4,5-b]quinoline-1,10-dione.

Particular compounds of the invention are those according to structural diagram I wherein R¹ is chloro; n is 1, and D is phenyl or pyridyl.

Other particular compounds of the invention are those according to structural diagram 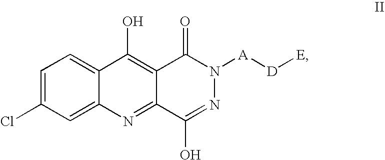
wherein A is (CH₂)ₙC≡C where n is selected from 1, 2 or 3; D is phenyl or pyridyl and E is halogen or hydrogen; with the proviso that said compound of structural diagram II is not 7-chloro-4-hydroxy-2-[3-(phenyl)prop-2-ynyl]-1,2,5,10-tetrahydropyridazino[4,5-b]quinoline-1,10-dione.

More particular compounds of the invention are those according to structural diagram II wherein A is (CH₂)ₙC≡C where n is 1, D is phenyl and E is halogen.

Other particular compounds of the invention are those according to structural diagram II wherein A is (CH₂)ₙC≡C where n is 1, D is pyridyl and E is halogen or hydrogen.

The most particular compounds of the invention are those exemplary compounds disclosed herein.

Other compounds useful in the methods and compositions of the invention are pharmaceutically-acceptable salts of compounds in accord with structural diagram I and tautomers of such a compounds.

In a second aspect the invention provides a method for the treatment of pain comprising administering to a subject suffering from pain a pain-ameliorating effective amount of any compound according to structural diagram I. 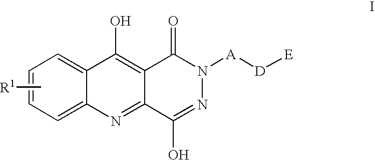
wherein: R¹ is halo; A is (CH₂), where n is a value selected from 1, 2 or 3; D is aryl or heteroaryl; E is hydrogen or halogen, or a tautomer or pharmaceutically-acceptable salt thereof.

A particular aspect of the invention provides a method for the treatment of neuropathic pain.

In particular embodiments the method comprises administering a pain-ameliorating effective amount of a compound according to structural diagram I, wherein R¹ is chloro; n is 1, and D is phenyl or pyridyl.

In other embodiment of the invention the method comprises administering a pain-ameliorating effective amount of a compound according to structural diagram II, 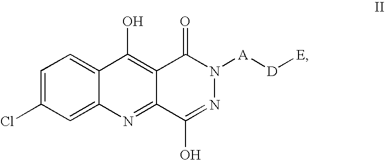
wherein A, D and E are as heretofore described.

In yet other embodiment of the invention the method comprises administering a pain-ameliorating effective amount of a compound according to structural diagram II wherein n is 1, and D is phenyl or pyridyl.

In further embodiment of the invention the method comprises administering a pain-ameliorating effective amount of a compound according to structural diagram II wherein, either D is phenyl and E is halogen, or D is pyridyl and E is hydrogen.

Still more particular embodiments of the invention are those where the method comprises treatment with an exemplary compound specifically disclosed herein.

Another aspect of the invention is a method for making compounds in accord with structural diagram I.

Yet other aspects of the invention are pharmaceutical compositions which contain a compound in accord with structural diagram I; the use of compounds in accord with structural diagram I for the preparation of medicaments and pharmaceutical compositions, and a method comprising binding a compound of the invention to the NMDA receptor glycine site of a warm-blooded animal, such as a human being, so as to beneficially inhibit the activity of the NMDA receptor.

### DETAILED DESCRIPTION OF THE INVENTION

Compounds of the invention are those within the scope of the generic description and particularly those compounds exemplified hereafter.

Suitable pharmaceutically-acceptable salts of compounds of the invention include acid addition salts such as methanesulphonate, fumarate, hydrochloride, hydrobromide, citrate, tris(hydroxymethyl)aminomethane, maleate and salts formed with phosphoric and sulphuric acid. In other embodiments, suitable salts are base salts such as an alkali metal salts for example sodium, alkaline earth metal salts for example calcium or magnesium, organic amine salts for example triethylamine, morpholine, N-methylpiperidine, N-ethylpiperidine, procaine, dibenzylamine, choline, N,N-dibenzylethylarmine or amino acids such as lysine.

Another aspect of the invention is a process for making compounds of the invention, comprising preparing a Boc-protected hydrazine, coupling said Boc-protected hydrazine and cyclizing the product according to the process of the following scheme to form a compound according to structural diagram I: 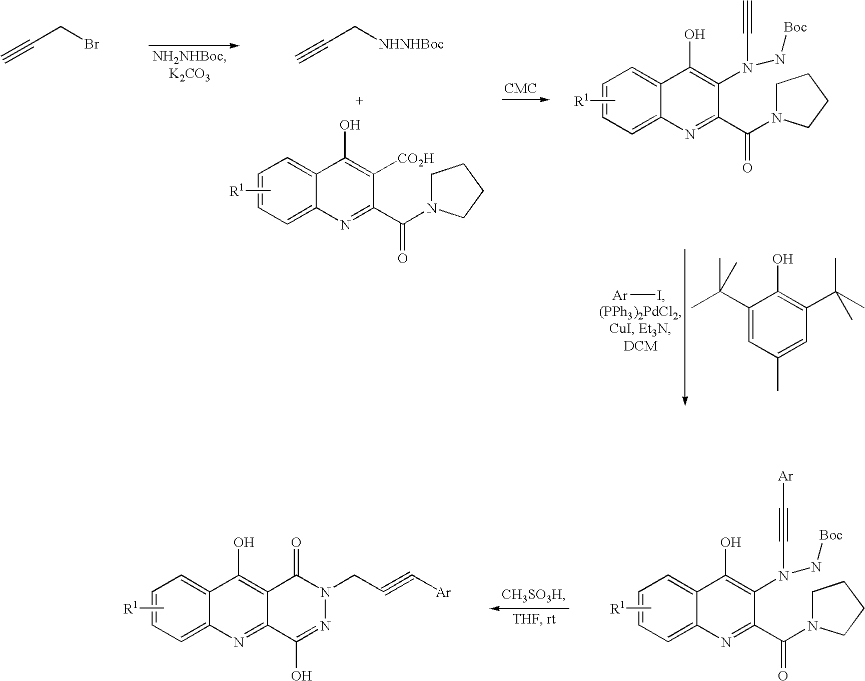
wherein:
CMC is 1-cyclohexyl-3-(2-morpholinoethyl)carbodiimide metho-p-toluenesulfonate;
the 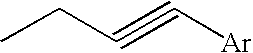
   moiety corresponds to the -A-D-E moiety of Structural diagram I, and
throughout the foregoing process R¹ is as defined for structural diagram I.

To use a compound of the invention or a pharmaceutically-acceptable salt thereof for the therapeutic treatment, which may include prophylactic treatment, of pain in mammals, which may be humans, the compound can be formulated in accordance with standard pharmaceutical practice as a pharmaceutical composition.

Suitable pharmaceutical compositions that contain a compound of the invention may be administered in conventional ways, for example by oral, topical, parenteral, buccal, nasal, vaginal or rectal administration or by inhalation. For these purposes a compound of the invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions. A preferred route of administration is orally by tablet or capsule.

In addition to a compound of the present invention a pharmaceutical composition of this invention may also contain one or more other pharmacologically-active agents, or such pharmaceutical composition may be simultaneously or sequentially co-administered with one or more other pharmacologically-active agents.

Pharmaceutical compositions of this invention will normally be administered so that a pain-ameliorating effective daily dose is received by the subject. The daily dose may be given in divided doses as necessary, the precise amount of the compound received and the route of administration depending on the weight, age and sex of the patient being treated and on the particular disease condition being treated according to principles known in the art. A preferred dosage regime is once daily.

A further embodiment of the invention provides a pharmaceutical composition which contains a compound of the structural diagram I as defined herein or a pharmaceutically-acceptable salt thereof, in association with a pharmaceutically-acceptable additive such as an excipient or carrier.

A yet further embodiment of the invention provide the use of a compound of the structural diagram I, or a pharmaceutically-acceptable salt thereof, in the manufacture of a medicament useful for binding to the NMDA receptor glycine site in a warm-blooded animal such as a human being.

Still another embodiment of the invention provides a method of binding a compound of the invention to the NMDA receptor glycine site of a warm-blooded animal, such as a human being, in need of treatment for pain, which method comprises administering to said animal an effective amount of a compound of structural diagram I or a pharmaceutically-acceptable salt thereof.

Definitions:

When used herein the term “halo” means fluoro, chloro, bromo and iodo.

When used herein the term “aryl” means an unsaturated carbon ring or a benz-derivative thereof. Particularly, aryl means phenyl, naphthyl or biphenyl. More particularly aryl means phenyl.

When used herein the term “heteroaryl” or “heteroaryl ring” means, unless otherwise further specified, a monocyclic-, bicyclic- or tricyclic-5-14 membered ring that is unsaturated or partially unsaturated, with up to five ring heteroatoms selected from nitrogen, oxygen and sulphur wherein a —CH₂— group can optionally be replaced by a —C(O)—, and a ring nitrogen atom may be optionally oxidized to form the N-oxide. Examples of such heteroaryls include thienyl, furyl, pyranyl, pyrrolyl, imidazolyl, pyrazolyl, thiazolyl, oxazolyl, isoxazolyl, pyridyl, pyridyl-N-oxide, oxopyridyl, oxoquinolyl, pyrimidinyl, pyrazinyl, oxopyrazinyl, pyridazinyl, indolinyl, benzofuranyl, benzimidazolyl, benzothiazolyl, quinolyl, isoquinolinyl, quinazolinyl, xanthenyl, quinoxalinyl, indazolyl, benzofuranyl and cinnolinolyl.

Generally in the methods, processes and examples described herein:
concentrations were carried out by rotary evaporation in vacuo;
operations were carried out at ambient temperature, that is in the range 18-26° C. and under a nitrogen atmosphere;
column chromatography (by the flash procedure) was performed on Merck Kieselgel silica (Art. 9385) unless otherwise stated;
yields are given for illustration only and are not necessarily the maximum attainable;
the structure of the end-products of the formula I were generally confirmed by NMR and mass spectral techniques, proton magnetic resonance spectra were determined in DMSO-d₆ unless otherwise stated using a Varian Gemini 2000 spectrometer operating at a field strength of 300 MHz; chemical shifts are reported in parts per million downfield from tetramethylsilane as an internal standard (δ scale) and peak multiplicities are shown thus: s, singlet; bs, broad singlet; d, doublet; AB or dd, doublet of doublets; t, triplet, dt, double of triplets, m, multiplet; bm, broad multiplet; fast-atom bombardment (FAB) mass spectral data were obtained using a Platform spectrometer (supplied by Micromass) run in electrospray and, where appropriate, either positive ion data or negative ion data were collected, in this application, (M+H)⁺ is quoted;
intermediates were not generally fully characterized and purity was in general assessed mass spectral (MS) or NMR analysis.

The following abbreviations and definitions when used, have the meanings, as follows:

| | |
|---|---|
| CDCl₃ | is deuterated chloroform; |
| CMC | is 1-cyclohexyl-3-(2-morpholinoethyl)carbodiimide |
| | metho-p-toluenesulfonate; |
| DCM | is dichloromethane; |
| DCU | is dicyclohexyl urea; |
| DHC | is 1,3-dicyclohexylcarbodiimide; |
| DMAP | is 4-(dimethylamino)pyridine; |
| DMF | is N,N-dimethylformamide; |
| DMSO | is dimethylsulphoxide; |
| m/s | is mass spectroscopy; |
| NMP | is N-methylpyrrolidinone; |
| NMR | is nuclear magnetic resonance; |
| p.o. | is per os; |
| THF | is tetrahydrofuran, and |
| t.i.d. | is three times daily. |

The examples and tests described herein are intended to illustrate but not limit the invention.

### EXAMPLES

### Example 1

### 7-Chloro-4-hydroxy-2-(3-(4-pyridyl)prop-2-ynyl)1,2,5,10-tetrahydropyridazino[4,5-b]quinoline-1,10-dione 1,3 methanesulfonate. (tert-Butoxy)-N-(pron-2-ynylamino)carboxamide

A 5 liter three-necked flask equipped with mechanical stirrer, thermometer and 500 mL dropping funnel with nitrogen inlet, was charged with powdered potassium carbonate (124.03 g, 0.8975 mol), tert-butyl carbazate (355.4 g, 2.692 mol), and 2700 mL of 9:1 THF:DMF. To this stirred slurry was added a solution of propargyl bromide (100 mL, 0.898 mole, of 80% in toluene), which was dissolved in 300 mL of 9:1 THF:DMF, over 1.5 hrs. The reaction was stirred at ambient temperature for 44 hrs. and the contents were then concentrated. The residue was partitioned between 1500 mL of DCM and 2000 mL water. The aqueous layer was extracted with DCM (2×500 mL) and the combined organics were washed once with 1000 mL water/200 mL brine and then several times with 400 mL water/100 mL brine. The organic layer was dried over Na₂SO₄ and concentrated to give a yellow oil. The product (365.4 g) was dissolved in 3000 mL of diethyl ether and was treated with 1500 mL of 1 N ethereal HCl over 3 hrs. The thick white solids which formed were filtered and washed with diethyl ether and the filtrates were concentrated to give the product as a yellow oil (136.02 g). The oil was applied to a column containing 2400 mL of silica wet-packed in hexane. Elution was performed with: hexane (2 L), 90:10 hexane:diethyl ether (2 L), 80:20 hexane:diethyl ether (2 L) and 70:30 hexane:diethyl ether (6 L). The product was obtained as a white solid (83.6 g, 54%). ¹H NMR (300 MHz, DMSO-d₆): δ 1.39 (s, 9H), 3.06 (s, 1H), 3.43 (s, 2H), 4.68 (s, 1H), 8.29 (bs, 1H).

N-[(tert-Butoxycarbonylamino][7-chloro-4-oxo-2-(pyrrolidinylcarbonyl)(3-hydroquinolyl)]-N-prop-2-ynylcarboxamide.

To a stirred suspension of 7-chloro-4-oxo-2-(pyrrolidinylcarbonyl)hydroquinoline-3-carboxylic acid (37.86 g, 0.118 mol) in THF (600 mL) was added CMC (100.0 g, 0.236 mol). The white suspension became bright yellow immediately. To this stirred suspension was added over 30 minutes a solution of (tert-butoxy)-N-(prop-2-ynylamino)carboxamide (20.93 g, 0.123 mol) in THF (250 mL) and the reaction was stirred at ambient temperature for 24 hrs. The reaction mixture was filtered and the collected solids washed with THF; the filtrates and washes were combined and concentrated. The residue was partitioned between 1500 mL DCM and 1500 mL water and the aqueous layer was extracted once with DCM. The combined organic layer/extract was washed once with brine, dried over MgSO₄ and concentrated. The residue was chromatographed over silica gel using the following eluants: CH₂Cl₂ (500 mL), 98:2 CH₂Cl₂/MeOH (2000 mL), 95:5 CH₂Cl₂/MeOH (4000 mL). The product was obtained initially as a foam which was then triturated with 220 mL of 1:1 hexane:diethyl ether to give the title compound as a white powder (46.57 g, 83%).

¹H NMR (300 MHz, DMSO-d₆): δ 1.42-1.18 (m, 9H), 1.86 (m, 4H), 3.44-3.36 (m, 6H), 4.20 (s, 1H), 7.50 (d, 1H, J=8.7 Hz), 7.64 (s, 1H), 8.18 (d, 1H, J=8, 7 Hz), 8.73 (br s, 1H), 12.90 (br s, 1H),

N-[(tert-Butoxy)carbonylamino][7-chloro-4-oxo-2-(pyrrolidinylcarbonyl)(3-hydroquinolyl)]-N-(3-(4-pyridyl)prop-2-ynyl)carboxamide.

A solution of N-[(tert-butoxy)carbonylamino][7-chloro-4-oxo-2-(pyrrolidinylcarbonyl)(3-hydroquinolyl)]-N-prop-2-ynylcarboxamide (0.832 g, 1.76 mmol), 4-iodopyridine (0.327 g, 1.59 mmol), dichlorobis(triphenylphosphine)-palladium(II) (23.8 mg, 0.03 mmol), copper(I) iodide (8.2 mg, 0.04 mmol), 2,6-di-tert-butyl-4-methylphenol (18.1 mg, 0.08 mmol) and triethylamine (0.6 mL, 0.44 g, 4.3 mmol) in anhydrous DCM (30 mL) under a nitrogen atmosphere was stirred at room temperature overnight. The reaction mixture was concentrated and the oily residue was purified by silica gel flash column chromatography (CH₂Cl₂:MeOH gradient 100:0 to 97:3) to give the title compound as an oil (0.93 g, 106%). MS (CI) m/z 550/552.

### 7-Chloro-4-hydroxy-2-(3-(4-pyridyl)prop-2-ynyl)1,2,5,10-tetrahydropyridazino[4,5-b]quinoline-1,10-dione 1,3 methanesulfonate

To a stirred suspension of N-[(tert-butoxy)carbonylamino][7-chloro-4-oxo-2-(pyrrolidinylcarbonyl)(3-hydroquinolyl)]-N-(3-(4-pyridyl)prop-2-ynyl)carboxamide (0.87 g, 1.59 mmol) in THF (80 mL) under nitrogen was added methanesulfonic acid (5.1 mL, 7.55 g, 78.6 mmol). The resulting dark orange solution was stirred at room temperature for 25.5 hr, during which time a precipitate formed. The precipitate was collected by filtration, washed with THF and air dried. The collected solids were suspended/stirred in methanol (10 mL) for 2 hr and then filtered and dried in vacuo overnight to provide the title compound as a light tan solid (396.2 mg, 66%), mp 238-241° C. ¹H NMR (300 MHz, DMSO-d₆): δ 2.32 (s, ˜3H) 5.06 (s, 2H), 7.45 (d, 1H, J=8.4 Hz), 7.66 (d, 2H, J=4.5 Hz), 8.04 (s, 1H), 8.15 (d, 1H, J=8.4 Hz), 8.71 (s, 2H), 12.0 (br s, 1H, exchangeable), 12.9 (br s, 1H, exchangeable). MS (CI) m/z 379/381. Calc'd. for C₁₉H₁₁ClN₄O₃.1.3CH₃SO₃H: C, 48.41; H, 3.24; N, 11.12. Found: C, 48.45; H, 3.26; N, 11.11.

### Example 2

### 7-Chloro-4-hydroxy-2-[3-(3-chlorophenyl)prop-2-ynyl]-1,2,5,10-tetrahydropyridazino[4,5-b]quinoline-1,10-dione.

N-[(tert-Butoxy)carbonylamino][7-chloro-4-oxo-2-(pyrrolidinylcarbonyl)(3-hydroquinolyl)]-N-(3-(3-chlorophenyl)prop-2-2-ynyl)carboxamide.

A solution of 3-chloroiodobenzene (0.303 g, 1.27 mmol), dichlorobis-(triphenylphosphine)palladium(II) (25 mg, 0.036 mmol) and copper(I) iodide (12.7 mg, 0.067 mmol) in chloroform (10 mL) was stirred at room temperature under argon for 5 minutes. To the resulting dark yellow solution was added N-[(tert-butoxy)-carbonylamino][7-chloro-4-oxo-2-(pyrrolidinylcarbonyl)(3-hydroquinolyl)]-N-prop-2-ynylcarboxamide (0.604 g, 1.28 mmol) and triethylamine (0.5 mL, 0.36 g, 3.6 mmol) and the resulting orange solution was refluxed under argon for 1.5 hr and then cooled to room temperature. The reaction mixture was poured into an excess of 1% aqueous hydrochloric acid and an additional 40 mL of chloroform was added. The chloroform layer was separated from the aqueous layer and washed with water (3×50 mL). The aqueous layer and aqueous washes were combined and extracted with ethyl acetate (3×20 mL). The combined chloroform and ethyl acetate washes were dried offer Na₂SO₄, filtered and concentrated in vacuo to provide an orange solid (0.81 g); purification by flash chromatography over silica gel (eluant: CH₂Cl₂:MeOH gradient 100:0 to 97:3) gave the title compound as a glassy yellow solid (0.28 g, 38%). MS (CI) m/z 583/585.

### 7-Chloro-4-hydroxy-2-[3-(3-chlorophenyl)prop-2-ynyl]-12,5,10-tetrahydropridazino[4,5-b]quinoline-1,10-dione

To a stirred suspension of N-[(tert-butoxy)carbonylamino][7-chloro-4-oxo-2-(pyrrolidinylcarbonyl)(3-hydroquinolyl)]-N-(3-(3-chlorophenyl)prop-2-ynyl)carboxamide (0.28 g, 0.48 mmol) in THF (20 mL) under nitrogen was added methanesulfonic acid (1.5 mL, 2.22 g, 2.31 mmol). The resulting dark yellow solution was stirred at room temperature for 3 days. The reaction mixture was poured into water (200 mL) and stirred for 0.5 hr. The precipitate which formed was collected by filtration and sonicated as a suspension in a minimum amount (5 mL) of methanol and then collected by filtration to provide, after drying in vacuo, the title compound as a yellow solid (132.8 mg, 67%), mp 220-222° C. ¹H NMR (300 MHz, DMSO-d₆): δ 4.98 (s, 2H), 7.39-7.66 (m, 5H), 8.03 (s, 1H), 8.15 (d, 1H, J=8.7 Hz), 11.99 (br s, 1H, exchangeable), 12.86 (br s, 1H, exchangeable). MS (CI) m/z 412/414. Calc'd. for C₂₀H₁₁Cl₂N₃O₃0.1 H₂O.1.1 CH₃OH: C, 56.41; H, 3.50; N, 9.35; Found: C, 56.28; H, 3.66; N, 9.51.

### Example 3

### 7-Chloro-4-hydroxy-2-[3-(4-chlorophenyl)prop-2-ynyl]-1,2,5,10-tetrahydropyridazino[4,5-b]quinoline-1,10-dione.

The title compound was prepared by the method described Example 2 from 4-chloroiodobenzene. Yield 89%; mp 251-252° C.; MS (CI) m/z 412/414; ¹H NMR (300 MHz, DMSO-d₆): δ 4.97 (s, 2H), 7.45 (s, 5H), 8.03 (s, 1H), 8.15 (d, 1H, J=8.7 Hz), 11.98 (br s, 1H, exchangeable), 12.86 (br s, 1H, exchangeable). Calc'd for C₂₀H₁₁Cl₂N₃O₃.0.5H₂O C, 57.03; H, 2.87; N, 9.98; Found: C, 57.23; H, 3.06; N, 9.75

### Example 4

### 7-Chloro-4-hydroxy-2-[3-(2-chlorophenyl)prop-2-ynyl]-1,2,5,10-tetrahydropyridazino[4.5-b]quinoline-1.10-dione

The title compound was prepared by the method described Example 2 from 2-chloroiodobenzene. Yield 32%; mp 255-257° C.; MS (CI) m/z 412/414; ¹H NMR (300 MHz, DMSO-d₆): δ 5.02 (s, 2H), 7.31-7.46 (m, 3H), 7.54 (d, 2H, J=7.8 Hz), 8.03 (s, 1H), 8.15 (d, 1H, J=8.7 Hz), 11.99 (br s, 1H, exchangeable), 12.87 (br s, 1H, exchangeable); Calc'd for C₂₀H₁₁Cl₂N₃O₃.0.4H₂O.0.55H₂O: C, 56.47; H, 3.23; N, 9.61; Found: C, 56.50; H, 3.19; N, 9.57

### Example 5

### 7-Chloro-4-hydroxy-2-(3-(3-pyridyl)prop-2-ynyl)-2,5,10-tetrahydropyridazino[4.5-b]quinoline-1,10-dione methanesulfonate

### 3-(3-pyridyl)prop-2-yn-1-ol

To a stirred solution of 1,2 dimethoxyethane:water (250 mL, 1:1), under nitrogen, was added potassium carbonate (29.5 g, 213 mmol), 3-bromopyridine (10 mL, 103.8 mmol), triphenylphosphine (3.24 g, 12.4 mmol), copper(I) iodide (1.96 g, 10.30 mmol), and 10% Pd/C (3.22 g, 3.02 mmole Pd). The reaction mixture was stirred for 20 min at room temperature and then propargyl alcohol (15 mL, 257.7 mmol) was added. The reaction mixture was heated at 80° C. for 14 hours and allowed to cool to room temperature. The reaction mixture was filtered through diatomaceous earth and the solids were then washed with water and ethyl acetate. The combined filtrate and washes were acidified to pH 2-3 using a 2 N HCl solution and the organic layer was removed in vacuo. The remaining aqueous phase was first extracted with toluene (3×50 mL) then its pH was adjusted to 7 by addition of potassium carbonate; the neutral aqueous solution was further extracted with ethyl acetate (3×500 mL). The ethyl acetate extracts were washed with water, dried over MgSO₄ and concentrated in vacuo. The residue gave the title compound as a yellow-brown product solid (10.07 g, 73% yield). ¹H NMR (300 MHz, DMSO-d₆); δ 4.34 (d, 2H, J=6 Hz); 5.44 (t, 1H, J=6 Hz); 7.41 (dd, 1H, J=8 Hz, J=5 Hz); 7.86(dt, 1H, J=8 Hz, J=2. Hz); 8.56 (d, 1H, J=5. Hz); 8.63 (s, 1H).

### 3-(3-Bromoprop-1-ynyl)pyridine hydrobromide

To a cold (−10° C.) stirred solution of 3-(3-pyridyl)prop-2-yn-1-ol (6.67 g, 50 mmol), in alcohol-free chloroform (100 mL) under nitrogen, was added PBr₃ (3 mL, 8.55 g, 31.6 mmol). The reaction mixture was stirred at −10° C. to 0° C. for 1.5 hr and then allowed to warm to room temperature. The upper clear yellow solution was transferred via a cannula to a new flask and the remaining brown reaction residue was washed with additional chloroform (50 mL) which was then combined with the first chloroform wash. Concentration of the combined chloroform washes gave the title compound as a pale yellow solid. On vigorous stirring of the residue (in 100 mL of chloroform) which did not dissolve in the chloroform washes, a white precipitate formed which was collected by filtration, washed with chloroform and air dried to provide the title compound as a pale yellow solid. The yield of the combined product batches was 9.90 g (70%). ¹H NMR (300 MHz, DMSO-d₆); δ 4.61 (s, 2H); 7.86 (dd, 1H, J=8 Hz, J=5 Hz); 8.39(dt, 1H, J=8 Hz, J=2 Hz); 8.83 (d, 1H, J=5. Hz); 8.99 (s, 1H).

(tert-Butoxy)-N-[(3-(3-pyridyl)prop-2-ynyl)amino]carboxamide.

To a stirred mixture of tert-butylcarbazate (20.07 g, 151.9 mmol) and potassium carbonate (6.25 g, 45.2 mmol) in dry DMF (50 mL) was added under nitrogen a solution of 3-(3-bromoprop-1-ynyl)pyridine hydrobromide (4.50 g, 16.25 mmol) dissolved in DMW (100 mL). The reaction mixture was stirred for five hours at room temperature and was filtered through diatomaceous earth to give an orange clear solution; the diatomaceous earth pad was washed with ethyl acetate (100 mL) and methanol (2×100 mL). The organic filtrate and washes were combined, poured into water (200 mL) and extracted with diethyl ether (3×150 mL) followed by ethyl acetate (2×200 mL). The organic extracts were combined, dried over MgSO₄, filtered and concentrated m/z vacuo to give a mixture of the product containing an excess of remaining tert-butylcarbazate. This solid (43 g) was dissolved in a diethyl ether:hexanes solution 1:3 (400 mL) which was washed with a saturated sodium bicarbonate solution (6×200 mL) to remove the tert-butylcarbazate. The organic material was then dried over MgSO₄, filtered and concentrated to give the product as a yellow oil (2.11 g, 52% yield).

1H NMR (300 MHz, DMSO-d₆); δ 1.39 (s, 9H); 3.73 (d, 2H, J=4 Hz); 4.89 (b, NH) 7.42 (dd, 1H, J=8 Hz, J=5 Hz); 7.84 (d, 1H, J=8 Hz); 8.42 (b, NH); 8.55 (d, 1H, J=3. Hz); 8.62 (d, 1H, J=4 Hz).

Dimethyl 7-chloro-4-hydroxyyquinoline-2,3-dicarboxylate:

A stirred mixture of methyl 2-amino-4-chlorobenzoate (2.50 g, 13.5 mmol) and dimethyl acetylenedicarboxylate (2.05 g, 14.4 mmol) in tert-butanol (22 ml) was refluxed for 7 hours under a nitrogen atmosphere. After adding additional dimethyl acetylenedicarboxylate (1.16 g, 8.13 mmol) and refluxing another 2.5 hours, the reaction mixture was allowed to cool to room temperature and potassium tert-butoxide (1.56 g, 13.9 mmol) was added in one portion. A precipitate formed and the resulting mixture was refluxed for 1.5 hours. The mixture was cooled to room temperature and filtered to separate the solids, which were washed with tert-butanol and diethyl ether. The solids were dissolved in water and acidified with 1 N sulfuric acid to form a precipitate. The resulting mixture was extracted with DCM and the combined extracts were washed with brine and water, dried over MgSO₄, filtered and concentrated to give a green solid. Recrystallization of this material from methanol provided the title compound (1.15 g, 47%) as an off-white solid, mp 232-233° C.; MS (CI):296 (M+H). Analysis for C₁₃H₁₀ClNO₅: Calc'd: C, 52.81; H, 3.41; N, 4.74; Found: C, 52.75; H, 3.47; N, 4.69.

### 3-Carbomethoxy-7-chloro-4-hydroxyquinoline-2-carboxylic Acid

To a stirred suspension of dimethyl 7-chloro-4-hydroxyquinoline-2,3-dicarboxylate (1.0 g, 3.38 mmol) in water (20 mL) was added an aqueous solution of sodium hydroxide (0.27 g, 6.75 mmol). Upon addition, the suspension dissolved. The reaction mixture was warmed to 60° C. for 1 hour. After this time the reaction was cooled to room temperature and acidified with concentrated hydrochloric acid. The product was then extracted into diethyl ether and ethyl acetate. The organic extracts were dried over MgSO₄, filtered and concentrated in vacuo to provide the title compound as a solid (900 mg). This material was purified by recrystallization employing an ethyl acetate/hexane co-solvent system to provide the title compound (571 mg, 60%) as a white solid mp 296° C. (dec); MS (CI)=238 (M+H). Analysis for Cl₂H₈NO₅Cl.0.45 CH₃CO₂CH₂CH₃.0.10H₂O: Calc'd: C, 51.30; H, 3.68; N 4.34, Found: C, 51.28; H, 3.62; N 3.97 ¹H NMR 8.22 (d, J=8.7 Hz, 1H), 7.92 (d, J=1.8 Hz, 1H), 7.28 (dd, J=8.7, 1.8 Hz, 1H), 3.90 (s, 3H).

### 3-Carbomethoxy-2-pyrrolidinocarbamide-7-chloro-4-hydroxyquinoline

To a suspension of 3-carbomethoxy-7-chloro-4-hydroxyquinoline-2-carboxylic acid (2.25 g, 8.0 mmol) in THF (20 mL) at ambient temperature under a N₂ atmosphere was added DHC (1.65 g, 8.0 mmol) and pyrrolidine (0.596 g, 8.4 mmol). The reaction was stirred at room temperature for 15 hours after which time the by-product urea was removed via filtration. The desired product was purified via flash column chromatography employing 5% methanol in chloroform to provide the title compound (2.52 g, 94.3%) as a tan solid, mp=215° C.; MS (CI): 335 (M+H). 300 MHz ¹H NMR (DMSO-d): δ 8.12 (d, J=8.7 Hz, 1H), 7.60 (d, 1H, J=1.8 Hz), 7.47 (dd, 1H, J=8.8, 2.0 Hz), 3.69 (s, 3H), 3.40-3.49 (m, 21), 3.27-3.33 (m, 2H), 1.80-1.96 (m, 4H).

### 7-Chloro-4-oxo-2-(pyrrolidinylcarbonyl)hydroquinoline-3-carboxylic Acid

To a suspension of 3-carbomethoxy-2-pyrrolidinocarbamide-7-chloro-4-hydroxy quinoline (2.52 g, 7.5 mmol) in de-ionized water (40 mL) was added dropwise a solution (20 mL) of an aqueous potassium hydroxide (882 mg, 15.75 mmol). Upon complete addition, the reaction was warmed to 60° C. After 3 hours, the reaction was filtered to remove a small amount of insoluble material. The filtrate was then acidified to pH=1 which yield a white precipitate. The solid was isolated by vacuum filtration, washed with water, and dried at 30° C. in vacuo for 16 hours. This provided the title compound (1.5 g, 64%) as a white solid, mp=225-8° C.; MS (CI): 321 (M+H). 300 MHz ¹H NMR (DMSO-d₆): δ 8.28 (d, J=8.8 Hz, 1H), 7.77 (s, 1H), 7.64 (d, 1H, J=8.7), 3.52-3.57 (m, 2H), 3.17-3.19 (m, 2H), 1.83-1.98 (M, 4H).

(tert-Butoxy)-N-{[7-chloro-4-oxo-2-(pyrrolidinylcarbonyl)(3-hydroquinolyl)]-N-(3-(3-pyridyl)prop-2-ynyl)carbonylamino}carboxamide.

To a stirred solution of 7-chloro-4-oxo-2-(pyrrolidinylcarbonyl)hydroquinoline-3-carboxylic acid (4.53 g, 14.12 mmol) in dry THF (85 mL) under nitrogen was added CMC (CMC, 13.23 g, 31.23 mmol) and N,N-dimethylaminopyridine (DMAP, 0.05 g, 0.4 mmol). The reaction mixture was stirred for 10 minutes and then a solution of (tert-butoxy)-N-[(3-(3-pyridyl)prop-2-ynyl)amino]carboxamide (3.49 g, 14.11 mmol) in dry THF (55 mL) was added. The reaction mixture was stirred at room temperature for 14 hours and filtered. The collected solids were washed with ethyl acetate several times and these washes were combined with the initial filtrate and concentrated in vacuo to give 5.46 g of a yellow solid. This material was flash chromatographed over silica gel using chloroform:methanol (gradient of chloroform to 5% MeOH:chloroform) as the eluant to give the title compound as a yellow solid (1.83 g, 24% yield).

### 7-Chloro-4-hydroxy-2-(3-(3-pyridyl)prop-2-ynyl)-1,2,5,10-tetrahydropyridazino[4,5-b]quinoline-1,10-dione methanesulfonate

To a stirred solution of (tert-butoxy)-N-{[7-chloro-4-oxo-2-(pyrrolidinylcarbonyl)(3-hydroquinolyl)]-N-(3-(3-pyridyl)prop-2-ynyl)carbonylamino}carboxamide (1.83 g, 3.33 mmol) in dry THF (80 mL) under nitrogen was added methanesulfonic acid (5 mL, 77 mmol). The reaction mixture was stirred at room temperature for 30 hours and iced water (100 mL) was added to form a precipitate. The resulting mixture was stirred for 2 hours and filtered to separate the solid which was washed successively with water, methanol and diethyl ether and then air dried. The filtrate was dried overnight at 45° C. in vacuo to give the title compound (0.645 g, 40%) as a yellow solid, mp 227-229° C. ¹H NMR (300 MHz, DMSO-d₆): δ 5.01 (s, 2H); 7.42-7.49 (m, 2H); 7.92 (d, 1H, J=7 Hz); 8.04 (s, 1H); 8.15 (d, 1H, J=9 Hz); 8.59 (d, 1H, J=4 Hz); 8.67 (s, 1H); 11.94 (br s, 1H, exchangeable); 12.81 (br s, 1H, exchangeable). Calc'd. for C₁₉H₁₁ClN₄O₃.1.2H₂O.1.0 CH₃SO₃H: C, 48.38; H, 3.53; N, 11.28. Found: C, 48.30; H, 3.51; N, 11.36.

### Example 6

### 7-Chloro-2-[3-(6-chloro(3-pyridyl))prop-2-ynyl]-4-hydroxy-2,5-dihydropyridazino[4,5-b]quinoline-1,10-dione.

The title compound was prepared from 2-chloro-5-iodopyridine by a procedure analogous to that described in Example 1. ¹H NMR (300 MHz, DMSO-d₆) δ 12.81 (br s, 1H); 11.96 (br s, 1H); 8.49 (d, 1H, J=1.8 Hz); 8.15 (d, 1H, J=8.7 Hz); 8.04 (d, 1H, J=1.5 Hz); 7.92 (dd, 1H, J=2.1, 8.1 Hz); 7.54 (d, 1H, J=8.1 Hz); 7.45 (dd, 1H, J=1.8, 8.7 Hz); 5.01 (s, 2H).

Tests for Biological Function:

Test A: Inhibition of Binding of [³H]-MDL105,519:

Binding of compounds to the NMDA receptor glycine site may be assessed by measuring the ability of test compounds to inhibit the binding of tritiated MDL105,519 to brain membranes bearing the receptor.

Rat Brain Membranes: The rat brain membranes used in the experiments were obtained from Analytical Biological Services Inc., and were prepared substantially in accordance with the method of B. M. Baron et al., J. Pharmacol. Exp. Ther. 250, 162 (1989). Briefly, fresh brain tissue including cerebral cortex and hippocampus from male Sprague Dawley rats was homogenized in 0.32 M sucrose and centrifuged at low speed to separate cellular membranes from other cellular components. The membranes were then washed 3 times using deionized water, followed by treatment with 0.04% Triton X-100. Finally, membranes were washed six times in 50 mM Tris citrate buffer, pH 7.4, and frozen at −80° C. until use.

[³]MDL105,519 (72 Ci/mmol) was purchased from Amersham. Cold MDL105,519 was purchased from Sigma/RBI. Binding assays were performed substantially in accordance with the protocol of B. M. Baron et al., J. Pharmacol. Exp. Ther. 279, 62 (1996), as follows. On the day of the experiment, brain membranes were thawed at room temperature and suspended in 50 mM tris acetate buffer, pH 7.4 (“TAB”). Seventy-five micro grams per milliliter protein (by using the BioRad dye) were used for competition binding. The experiments were carried out using 96-well plates. Membranes were incubated with 20 mL of compounds of various concentrations and 1.2 nM [³H]MDL105,519 for 30 minutes at room temperature in a total volume of 250 μL. Non specific binding was determined by using 100 μM of unlabeled MDL105,519. The unlabeled MDL105,519 and compounds were dissolved as 12.5 mM stock solutions in DMSO. Final DMSO concentration in each well was kept below 1%, which concentration was found not to alter the binding results. After incubation, unbound [³H]MDL105,519 was removed by filtration onto GF/B Unifilter plates using a Packard harvester. Filters were washed four times with ice cold TAB (total of 1.2 mL buffer). The plates were dried overnight at room temperature and bound radioactivity was measured on a Packard TopCount after the addition of 45 μL per well of the MICROSCINT O.

Human Brain Membranes: Human brain membranes were obtained from Analytical Biological Services Inc., and assays were performed as described for rat membranes.

Data analysis: Data was analyzed using a Microsoft Excel spreadsheet and GraphPad Prizm software and potency of compounds is expressed as the Ki (nM).

Test B: Formalin Test:

The Formalin test is an assay that assesses the capacity of a compound to inhibit formalin-induced nociceptive behaviors in rats (D. Dubuisson, et al., Pain 4, 161-174 (1977); H. Wheeler-Aceto et al., Psychopharmacology 104, 35-44 (1991); T. J. Coderre, et al., Pain 54, 43-50 (1993)). In the test, two distinctive phases of formalin-induced behaviors are observed. A first phase response, caused by acute nociception to the noxious chemical (formalin) injected into the paw, occurs between zero and five minutes. A quiescent period of 5 to 15 min post injection follows. After the quiescent period a second phase response, caused by sensitization of the central neurons in the dorsal horn, occurs after 15 minutes and lasts up to 60 minutes. Sensitization of the central neurons in the spine augments a noxious afferent input and causes a stronger pain barrage to be transmitted to the brain. Therefore, inhibition of the second phase response indicates a central mechanism of drug action.

The procedure for the formalin test may be performed as follows: male rats are placed in a plexiglass chamber and observed for 30-45 min. to observe their baseline activity. Animals would either be pretreated with vehicle or with different doses of a test compound and are dosed with vehicle or test compound three hours prior to injection of 0.05 mL of sterile 1% formalin under the dorsal skin of a hind paw. The number of paw flinches (responses) during the first phase (0-5 min.) and the second phase (20-35 min.) are scored and recorded. Flinch response can be compared with the mean score of a saline control group and calculated as percentage inhibition. The ED₅₀ is the dose of compound which produced 50% inhibition of nociceptive response in the first or second phase response.

% inhibition of nociceptive response can be calculated as:

$$100 \times \frac{( number of responses in vehicle group - \\ number of responses in compound group )}{( number of responses in vehicle group )}$$

Student's t-test can be used for statistical analysis to determine the significance of compound effects.

Test C: Neuropathic Pain Model (Chronic Constriction Injury):

The anti-hyperalgesic properties of a compound may be tested with the Chronic Constriction Injury (“CCI”) model. The test is a model for neuropathic pain associated with nerve injuries that can arise directly from trauma and compression, or indirectly from a wide range of diseases such as infection, cancer, metabolic conditions, toxins, nutritional deficiencies, immunological dysfunction, and musculoskeletal changes. In the model a unilateral peripheral hyperalgesia is produced in rats by nerve ligation (G. J. Bennett, et al., Pain 33, 87-107 (1988)).

Procedurally, Sprague-Dawley rats (250-350 g) are anesthetized with sodium pentobarbital and the common sciatic nerve exposed at the level of the mid thigh by blunt dissection through the biceps femoris. A section of nerve (about 7 mm), proximal to the sciatic trifucation, is freed of tissue and ligated at four positions with chromic gut suture, with the suture tied with about 1 mm spacing between ligatures. The incision is closed in layers and the animals allowed to recuperate. Thermal hyperalgesia is measured using a paw-withdrawal test (K. Hargreaves, et al., Pain 32, 77-88 (1988)). To perform the test, animals are habituated on an elevated glass floor and a radiant heat source aimed at the mid-plantar hindpaw (sciatic nerve territory) through the glass floor with a 20 second cut-off to prevent injury to the skin. The latencies for the withdrawal reflex in both hind paws are recorded.

In this test, paws with ligated nerves show shorter paw withdrawal latencies compared to the unoperated or sham operated paws. Responses to test compounds are evaluated at different times after oral administration to determine the onset and duration of compound effect. When performing the test, groups of CCI rats would receive either vehicle or the test compound orally three times daily for 5 days. Paw withdrawal latencies can be measured each day 10 min. before and two or three hr. after the first daily dose. Compound efficacy is calculated as mean percentage decrease of hyperalgesia compared to a vehicle-treated group. Compound potencies may be expressed as the minimum effective dose (MED) in mg/Kg/day that yields a % decrease in hyperalgesia that is statistically significant, where the % anti-hyperalgesic effect may be calculated as follows:

$$\frac{( Mean of vehicle group - Mean of compound group )}{( Mean of vehicle group )} \times 100$$

Data analysis can be performed by the multiple means comparison (Dunnett's test).

Table 1 shows the results from Test A for the exemplified compounds of the invention.

TABLE 1

| | Test A |
|---|---|
| | Ki (nM) |
| Ex. 1 | 67.6 |
| Ex. 2 | 7.86 |
| Ex. 3 | 86.6 |
| Ex. 4 | 113 |
| Ex. 5 | 14 |
| Ex. 6 | 106 |

1. Any compound according to structural diagram I; 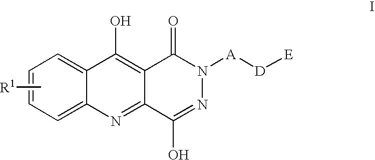
wherein:
R¹ is halo;
A is (CH₂)ₙC≡C where n is a value selected from 1, 2 or 3;
D is aryl or heteroaryl;
E is hydrogen or halogen,
or a tautomer or pharmaceutically-acceptable salt thereof,
with the proviso that said compound of structural diagram I is not 7-chloro-4-hydroxy-2-[3-(phenyl)prop-2-ynyl]-1,2,5,10-tetrahydropyridazino[4,5-b]quinoline-1,10-dione.

2. A compound according to claim 1, wherein:
R¹ is chloro;
n is 1, and
D is phenyl or pyridyl.

3. A compound of claim 1, according to structural diagram II,

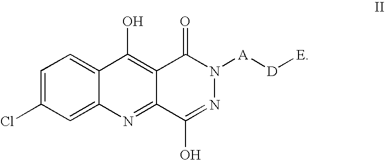

4. A compound according to claim 3, wherein:
n is 1, and
D is phenyl or pyridyl.

5. A compound according to claim 4, wherein:
D is phenyl and
E is halogen.

6. A compound according to claim 4, wherein:
D is pyridyl and
E is hydrogen or halogen.

7. A compound according to claim 1, selected from:
7-chloro-4-hydroxy-2-[3-(3-chlorophenyl)prop-2-ynyl]-1,2,5,10-tetrahydropyridazino[4,5-b]quinoline-1,10-dione;
7-chloro-4-hydroxy-2-(3-(4-pyridyl)prop-2-ynyl)-1,2,5,10-tetrahydropyridazino[4,5-b]quinoline-1,10-dione;
7-chloro-4-hydroxy-2-[3-(4-chlorophenyl)prop-2-ynyl]-1,2,5,10-tetrahydropyridazino[4,5-b]quinoline-1,10-dione;
7-chloro-4-hydroxy-2-[3-(2-chlorophenyl)prop-2-ynyl]-1,2,5,10-tetrahydropyridazino[4,5-b]quinoline-1,10-dione;
7-chloro-4-hydroxy-2-(3-(3-pyridyl)prop-2-ynyl)-1,2,5,10-tetrahydropyridazino[4,5-b]quinoline-1,10-dione, and
7-chloro-2-[3-(6-chloro(3-pyridyl))prop-3-ynyl]-4-hydroxy-2,5-dihydropyridazino[4,5-b]quinoline-1,10-dione.

8. A method for treating a subject suffering from pain comprising administering a pain-ameliorating effective amount of a compound according to structural diagram I, 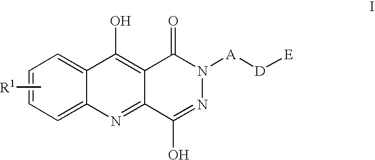
wherein:
R¹ is halo;
A is (CH₂)ₙC≡C where n is a value selected from 1, 2 or 3;
D is aryl or heteroaryl;
E is hydrogen or halogen,
or a tautomer or pharmaceutically-acceptable salt thereof.

9. A method according to claim 8, wherein in a compound according to structural diagram I:
R¹ is chloro;
n is 1, and
D is phenyl or pyridyl.

10. A method according to claim 8, comprising administering a pain-ameliorating amount of a compound according to structural diagram II,

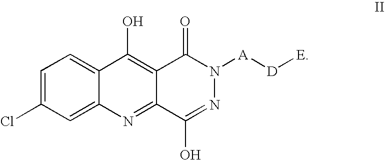

11. A method according to claim 9, wherein in a compound according to structural diagram II:
n is 1, and
D is phenyl or pyridyl.

12. A pharmaceutical composition comprising a pain-ameliorating effective amount of a compound according to structural diagram I 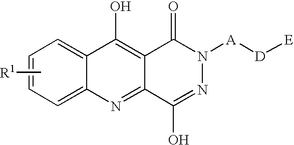
wherein:
R¹ is halo;
A is (CH₂)ₙC≡C where n is a value selected from 1, 2 or 3;
D is aryl or heteroaryl;
E is hydrogen or halogen,
or a tautomer or pharmaceutically-acceptable salt thereof,
together with a pharmaceutically-acceptable excipient or diluent.
